(19) United States
(12) Patent Application Publication
(10) Pub. No.: US 2005/0239712 A1
(43) Pub. Date: Oct. 27, 2005

(54) Pharmaceutically active compound

(76) Inventor: Charles Oswald Mills, Birmingham (GB)

Correspondence Address: BELL & ASSOCIATES, 416 FUNSTON ST., SUITE 100, SAN FRANCISCO, CA, 94118, US

(21) Appl. No.: 11/082,164

(22) Filed: Mar. 16, 2005

Related U.S. Application Data
(63) Continuation of application No. 10/333,525, filed on Sep. 18, 2003, now abandoned.

(30) Foreign Application Priority Data
Jul. 21, 2000 (GB) GB 00 17822.8

(51) Int. Cl. A61K 38/05; A61K 31/704; A61K 31/7072; A61K 31/7076; C07J 43/00

(52) U.S. Cl. 514/18; 514/26; 514/174; 530/331; 536/6.1; 540/107

(57) ABSTRACT

A liver-targeting active compound having the general formula (I): 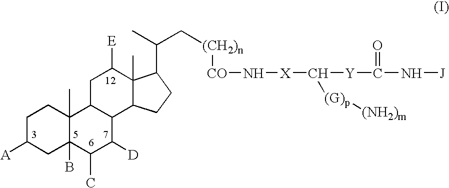
wherein A is α-OH or β-OH, B is α-H or β-H, C is —H, α-OH or β-OH, or B and C together form a double bond, D is —H, α-OH or β-OH, E is —H, α-OH or β-OH, -G- is a side chain moiety, —NH-J is selected from (i) a residue of an amino group-containing active compound wherein said —NH— group is provided by said amino group of the active compound, and (ii) a residue of an active compound to which an amino group has been added wherein said —NH— group is provided by said added amino group; each of X and Y independently represents a single bond, —(CH,₂)_{z}— (where z is 1 to 8), —O— or —S—; n is 0 or 1; m is 0 or 1; and p is 0 or 1; provided that, when —NH-J is (i), m is 1. A method of producing such a compound is also disclosed.

This invention relates to a pharmaceutically active compound and is more particularly concerned wit a liver-targeting pharmaceutically active compound.

Kramer W et al, J. Biol. Chem.; Vol 287: 18598-18604, 1992 and EP-B-0417725 discloses, inter alia, chlorambucil as a model drug to link to the bile acid at its 3α-OH position in the steroid ring to form an ester bond between the carboxyl group and the 3α-hydroxyl group of the bile acid.

Likewise, EP-A-0232788 and U.S. Pat. No. 4,793,949 discloses cancer drugs in which an N-halo alylcarbamoyl group is bound to the 3-OH position in the steroid ring.

Marin et al, Int. J. Cancer, 78: 346-352, 1998 and Macias et al, J. Lipid Res.; 39: 1792-1798, 1998 report the synopsis of cis plain conjugated to glycocholic acid via the carboxyl group of the glycine moiety of glycocholic acid with the result that one of the chloride atoms in cis platin was lost. The two chloride atoms in cis platin are very important in the potency of the drug as they form bifunctional adducts with DNA.

Manoharan et al, Annals of the New York Academy of Sciences, New York, vol. 660, 1992, pages 306-309, XP002162255, disclose the conjugation of cholic acid to oligonucleotide DNA phosphodiesters, phosphodiester RNA mimics and phosphorothioates using an amino linker to improve the bioavailabilty of antisense oligonucleotides. Thus, there is no amide bond between the and the pharmaceutically active moiety as in the present invention.

Stephen et al 1992, Biochem. Pharmacol.; 43: 196-1974 report the synthesis of drug-bile acid complex. The thyroid hormone L-T₃ was liked to the bile acid via an amide linkage between the C₂₅ carboxyl group of cholic acid and the α-ammo group of the alanine side-chain of L-triiodothyronine. Linkage of the drug with the bile acid resulted in loss of the amino of the drug which is considered be important in the potency of the drug.

C. O. Mills et al, Biochimica et Biophysica Acta, 115 (1991), pages 151-156, (see also WO 99/07325) disclose fluorescent bile salts based on cholyl-lysyl-fluorescein where the lysyl group is linked to the cholyl group by an amide linkage including the α-amino group of the lysine molecule, whilst the fluorescein group is linked to the ε-amino group of the lysine molecule via an isothiocyanate group. C. O. Mills et al (supra) note the similarity in biliary output and hepatic exaction between cholyl-lysyl-fluorescein and the natural bile acid cholylglycine and suggests that both compounds are handled in a similar fashion. One of the conclusions by C. O. Mills et al is that the greater biliary excretion and hepatic extraction of cholyl-lysyl-fluorescein relative to free fluorescein suggests that conjugation with a bile salt may be an efficient way of targeting compounds to the liver, although no specific teachings in this respect are given.

M. J. Monte et al, Journal of Hepatology, 1999; 31:521-528 disclose the use of bile acids as molecules for shutting cytostatic drugs toward liver tumours, and describe an anti-tumoral compound obtained by binding cisplatin to the carboxylate group of glycocholic acid to produce cis-diamminechlorocholylglycinate platinum(II).

It is an object of the present invention to provide a liver-targeting pharmaceutically active compound wherein there is a strong bond between a liver-targeting moiety and a pharmaceutically active moiety of the compound without unduly affecting the efficacy of the pharmaceutically active moiety, whereby it may be possible to target the latter to an intracell organelle of a liver cell.

According to the present invention, there is provided a liver-targeting pharmaceutically active compound having the general formula (I): 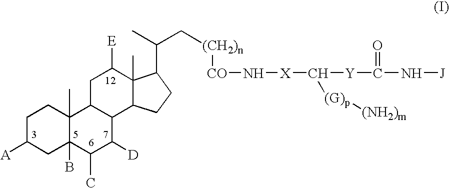
wherein A is α-OH or β-OH, B is α-H or β-H, C is —H, α-OH or β-OH, or B and C together form a double bond, D is —H, α-OH or β-OH, E is —H, α-OH or β-OH, -G- is a side chain moiety, —NH-J is selected from (i) a residue of an amino group-containing pharmaceutically active compound wherein said —NH— group is provided by said amino group of tie pharmaceutically active compound, and (ii) a residue of a pharmaceutically active compound to which an amino group has been added wherein said —NH— group is provided by said added amino group; each of X and Y independently represents a single bond, —(CH₂)_{z}— (where z is 1 to 8), —O— or —S—; n is 0 or 1; m is 0 or 1; and p is 0 or 1, provided that, when —NH-J is (i), m is 1.

The amide bond linking J to the remainder of the molecule is strong so that it is not cleaved in vivo. This represents a considerable departure from previous proposals which are based around providing a delivery vehicle for the drug based on a bile acid which is readily cleaved in vivo from the drug. Thus the present invention relies upon the provision of an amino group (or other group which can provide the —NH— group of —NH-J) associated with pharmaceutically active compound.

In the case where the pharmaceutically active compound inherently contains an amino group which provides the —NH— group of —NH-J (e.g. as in doxorubicin), it is essential for m to be 1 so at the group which is attached to the remainder of the molecule via the optional group -(G)ₚ- provides the function of the bound (and therefore non-functional) amino group of said pharmaceutically active compound. In this case, it is preferred for G and Y to be chosen so that the NH₂ group on the resultant side chain can be physically close to the —NH— group of —NH-J, whereby to mimic the effect of the unbound amino group normally present on the unconjugated pharmaceutically active compound.

In the case of a pharmaceutically active compound which does not have an —NH— group, such a group is added at an appropriate position in the molecule. In the case where the —NH— group is provided by an amino group added to a pharmaceutically active compound (e.g. tamoxifen), then there is no need for there to be a side chain amino group, and so m can be 0 in this case. In this latter case, the pharmaceutically active compound itself may or may not inherently contain amino group, but if it does then, since it does not take part in the link between such compound and the bile acid moiety, it is available to perform its required function.

The reside —NH-J may be based on any pharmaceutically active compound selected from antibiotics, diuretics, peptides, antiviral drugs, anticancer drugs, liver-treatment drugs, antihypertensive drugs, renin inhibitors, prolyl hydroxylase inhibitors, interferon inducers, DNA antisense/sense and ribosymes. Alternatively, —NH-J may be based on peptides, proteins or nucleotides (RNA or DNA).

For example, —NH-J may be based on doxorubicin; epirubicin; mitoxantrone; methotrexate; tamoxifen; mitomycin C; fluorouracil; cytarabine; thioguanine; acyclovir; ganciclovir; amphotericin; primaquine; ursodeoxycholyllysylcysteine; ursodeoxycholyllysylcysteic acid; ursodeoxycholyllysylmethionine; ursodeoxycholyllysyl-glntathione-(reduced); ursodeoxycholyllysylmethionine sulfone; amethopterin; arabinosyl-cytosine; L-cysteic acid; cysteine; L-cysteine sulphinic acid; N-acetylcysteine; methionime; methionine sulphone; methionine sulphoxide; L-glutathione; S-adenosyl-homocysteine; S-adenosyl-methionine; 8-aminoquinoline; tilorone(2,7-bis[2-(diethylamino)ethoxy]-9H-fluoren-9one); tilorone analogs such as tilorone dihydrochloride, 3,6- bis[2-(dimethylamino)ethoxy]-9H-xanthen-9-one dihydrochloride, 2,7-bis[dimethylaminoacetyl]-9H-xanthene dihydrochloride hydrate, 2,8-bis[dimethylaminoacetyl]-dibenzothiophene dihydrochloride hydrate and 2,8-bis[dimethylaminoacetyl]-dibenzofuran dihydrochloride hydrate; and melphalan(4-(bis[2-chloroethyl]amino)-L-phenylalanine).

Thus, the compound of the present invention are primarily concerned with the delivery to the liver of active compounds which are advantageously targetted to the liver. These can be categorised as follows:
1. Compounds for targeting liver-treatment drugs.
2. Compounds for targeting drugs for treating bile duct disorders.
3. Compounds for targeting protective agents (e.g. radio-protective agents) which is not a treatment in its own right.
4. Compounds for targeting an enzyme design for the local activation of another prodrug (e.g. by administering an inert produg which is locally activated in the liver by the targeted enzyme). This has the potential advantage that the drug might not be rapidly cleared because it is not linked to the bile acid moiety
5. Compounds for targeting an agent which actively metabolised in the liver to form an active which is generally available.

In the case of the side chain moiety, -G- may be —(CH₂)_{q}— (where q is 1 to 8, preferably 1 to 5, more preferably 3 to 5, and most preferably 4), or it may be —O— or —S—. Moiety -G- will normally only be present (i.e. when p=1) when there is a side chain amino group on the moiety linking the pharmaceutically active compound to the liver-targeting bile acid moiety (i.e. when m=1). In the case where p=1 and -G- is —O—, —S—, or —(CH₂)_{q}— (when q is 3 to 5), it is preferred for Y to represent a single bond.

The steroid moiety in the compound of the general formula (I) may be based on cholic acid, chenodeoxycholic acid, deoxycholic acid, hyodeoxcholic acid, hyocholic acid, α-, β-, or ω-muricholic acid, a nor-bile acid, lithocholic acid, 3β-hydroxycholenoic acid, ursodeoxycholic acid, allocholic acid (5α-cholan-24-oic acid), or the like.

Conjugated cholic acid is relatively hydrophilic and therefore not avidly taken up by intracellular organelles. Thus, it has a rapid hepatocellular transport. Conjugated deoxycholic acid, on the other hand, is less hydrophilic than cholic acid and penetrates cells more and therefore has a relatively slower hepatoceullar transport. It has apoptotic properties and is taken up by cholangiocytes (hepatocytes). Hence when conjugated to antitumour drugs deoxycholic acid may be targeted to cholangiocarcinoma (hepatocellular carcinoma)

Conjugated lithocholic acid is the most hydrophobic of the bile salts and therefore is the most cell-penetrating. It is taken up by the cell nucleus and therefore, when conjugated to drugs, may target the drug to the cell nucleus.

Conjugated ursodeoxycholic acid is strongly hydrophilic and therefore less cell-penetrating. Its hepatocellular transport is intermediate to that of cholic acid and lithocholic acid. Ursodeoxycholate has anticholestatic properties so, when liked to agents known to affect anticholestatic properties, it may enhance their anticholestatic potency via a synergistic or additional mechanism.

Also according to the present invention, there is provided a method of preparing a compound of the general formula (I) as defined above, comprising the step of reacting a compound of the general formula (II): 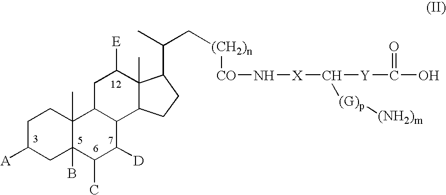
(wherein A, B, C, D, E, G, n, m and p are as defined above) with a pharmaceutically active compound having an amino group, so as to form an amide linkage between the carboxyl group in the compound of the general formula (II) and the amino group of the pharmaceutically active compound.

A preferred compound in accordance with the present invention is of the general formula (III): 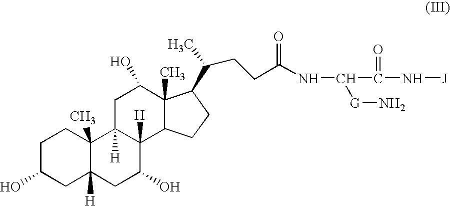
where G and J are as defined above.

The present invention will now be described in further detail:

### EXAMPLE 1

### Synthesis of Cholyl-Lysyl-Doxorubicin (VIII)

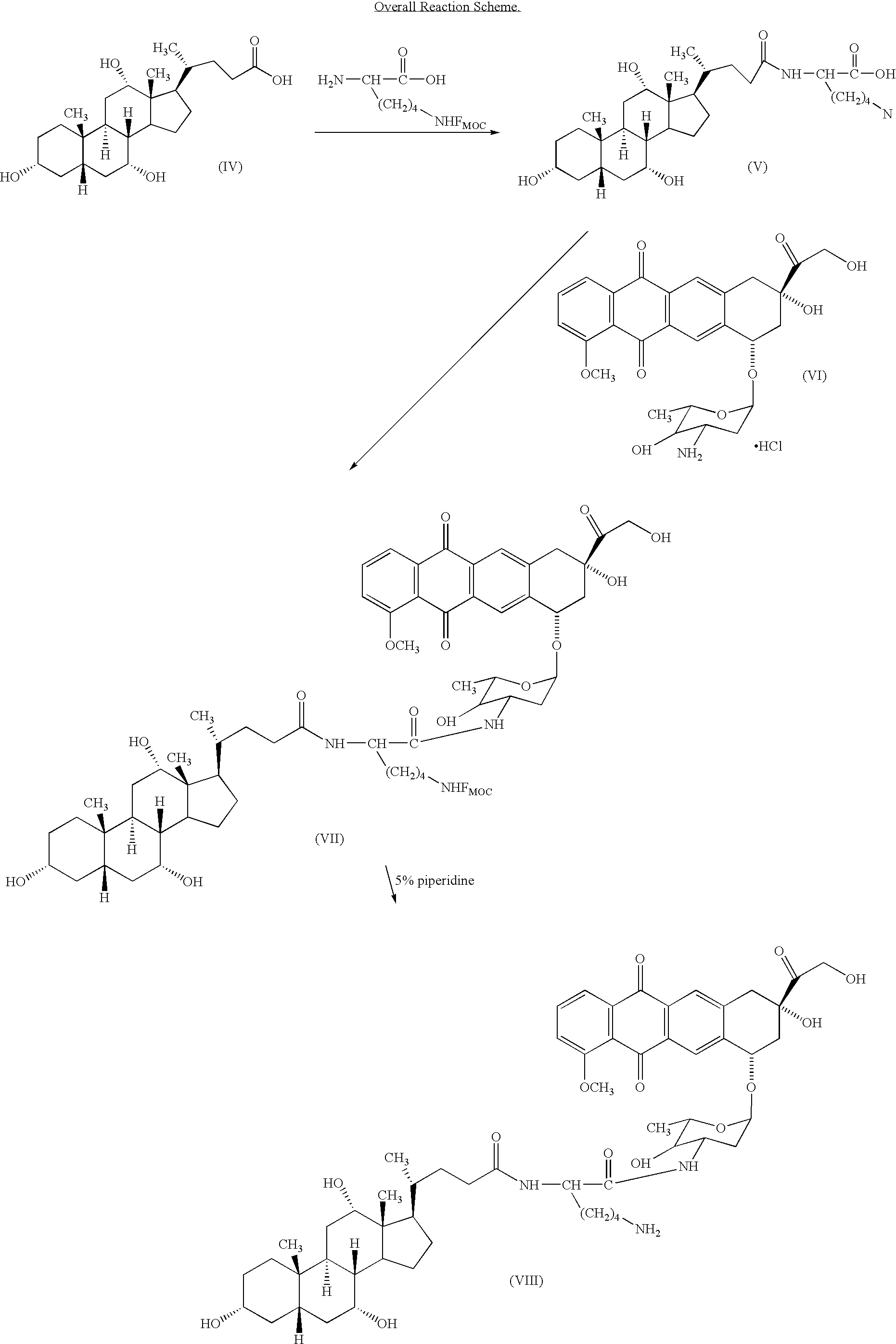

### Step 1—Preparation of Cholyl-lysine-N-ε-F_{MOC} (V)

Anhydrous acetone (10 cm³) and triethylamine (500 μl, 3.59 mmol) were added to Compound (IV), cholic acid, (1.46 g, 3.573 mmol) at −5° C. with stirring in a reaction vessel for 20 min followed by dropwise addition of isobutylchlorocarbonate (0.534 ml, 1.3 g, 4.56 mmol) whilst maintaining the temperature between −5° C. to −10° C. Twenty minutes after addition, the reaction was vigorously stir for a further 30 min at 15° C. Sodium hydroxide (0.153 g, 3.825 mmol) in water (8 cm³, 8 g) was added to N-ε-F_{MOC}-L-lysine (3.568 mmol, 1.314 g) in a 25 ml conical flask. Mixing was commenced and continued until a clear solution was formed (^{˜}20 min). The solution was quickly added to the cholic acid mixture above followed by vigorous stirring initially at +4° C. for 10 min further stirring at room temperature for a further 50 min .

1M HCl (4.43 cm³ was to the reaction mixture to give a final pH of about 2. The acidified solution was then extracted with ethyl acetate (3×50 ml) in a separating . The combined ethyl acetate extracts were filtered and washed with distilled water (pH 2) and then dried with sodium sulphate followed by evaporation to dryness in a rotary evaporator at 40° C. to give Compound (V), cholyl lysine-N-ε-Fmoc, as a white solid. Purity 90% yield 97%. Purther purification was by TKC.

### Step 2—Conversion to Compound (VIII)

Compound (VI), doxorubicin hydrochloride, (24 mg, 40 μmol) and triethylamine (6 μl, 40 μmol) were suspended in 300 μl N,N-dimethylformamide (DMF). Compound (V), cholyl-lysyl-F_{MOC}, (34 mg, 40 μmol) dissolved in 200 μl DMF was added. After cooling to 0-2° C., 1-hydroxybenzotriazole (6 mg, 40 μmol) were added followed by dicyclohexyl-carbodiimide (8 mg, 40 μmol) in 100 μl DMP. The mixture was stirred for 30 minutes at 0° C. and for 18 h at room temperature (21° C. in the dark. The DMF solution formed was acidified with 100 μl dilute ethanoic acid solution (1 ml glacial acetic acid in 10 ml distilled water). The resultant mixture was centrifuged, and the supernatant liquor was collected. Diethyl ether was added to form a gummy precipitate, followed by addition of 200 μl methanol and then concentration under vacuum. An additional 200 μl methanol was added, and Compound (VII), cholyl-lysyl(F_{MOC})doxorubicin was precipitated from the methanolic solution by dropwise addition of diisopropyl ether. The cholyl-lysyl(F_{MOC})doxorubicin was washed three times with diethyl ether and thoroughly dried in a vacuum for 20 minutes. Cholyl-lysyl(F_{MOC})doxorubicin was produced in 98% yield with a puriity of 96% by TLC.

Mild cleavage of cholyl-lysyl(F_{MOC})doxorubicin was effected using 5% piperidine (200 μl) and incubated for 10 minutes at room temperature. The reaction was stopped by addition of 20 μL 0.5M HCl followed by addition of diisopropylether to produce Compound (VIII), cholyl-lysyl-doxorubicin in ≧96% yield and ≧94% purity by TLC.

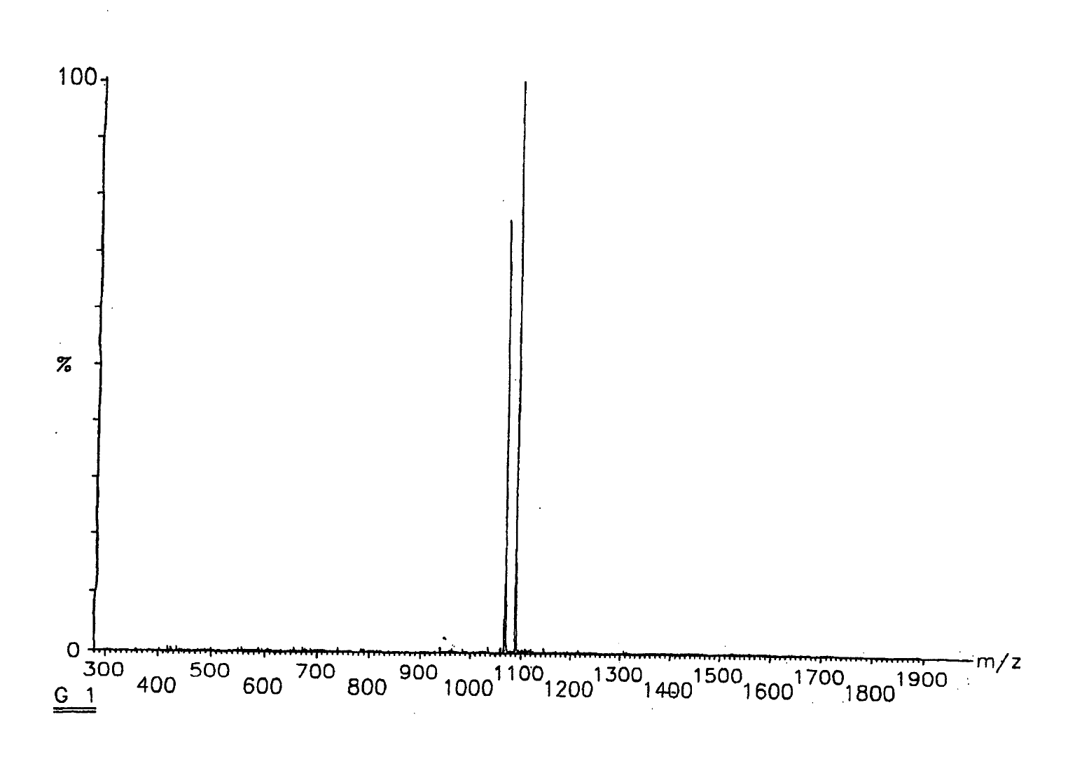

The compound (VIII) has a molecular mass on 1062.5 as determined by mass spectrometry (see FIG. 1 which is a mass spectrograph showing the mass of Compound (VIII) as well as that of a sodium adduct thereof (molecular mass=1084.5), the carbon-13 isotope (molecular mass=1085.4) and the carbon-14 isotope (molecular mass1086.4). Compound (VIII) has so far been shown to be soluble in water, methanol and ethanol, and insoluble in non-polar solvents such as ethoxyethane.

In Vitro Toxicity Tests

Cholyl-lysyl-doxorubicin (VIII) synthesised as described above was subjected to cytotoxicity tests in comparison with free doxorubicin and free cholate using in situ end labelling which enables semi-quantitative measure of apoptosis an necrosis.

HepG2 cells (Cobra American Type Culture Collection) in 24 well plates were used. Doxorubicin, cholate or cholyllysyldoxonubicin (in various amounts up to 0.86 μM) was added in triplicate and incubated for 4 h. The cells were trypsinized off from the well plates, cytospun, and then frozen.

In order to measure cell viability, sections were warmed up to room temperature for 30 minutes, then fixed in acetone for 10 minutes, and then wax was introduced around cells.

An in situ end labelling (ISEL) positive mixture was made up which included buffer (1 ml), dATP (1 μl), dCTP (1 μl), dGTP (1 μl), DiglldUTP (1 μl) and Klenow DNA polymerase (4 μl), and an in situ end labelling negative mixture of buffer (1 ml), dATP (1 μl) dCTP (1 μl), dGTP (1 μl), Digll, 2 dUTP and water (4 μl) was also made up. To each cytospin and coverslip was added 60 μl of the ISEL positive mixture or negative mixture. These were then floated in a water bath at 37° C. for 1 h. The coverslips were then removed and washed 3×5 mins in distilled water, followed by washing in TBS pH 7.5 for 5 mins. Anti digoxigen Alkaline phosphatase diluted 1:200 TBS was added and incubated for 1 h at room temperature and then washed in TBS pH 7.5 for 2.5 mins, followed by further washing in TBS pH 8.2 for another 2.5 mins. A substrate mixture was then made up to include naphthol phosphate (10 mg), NN-dimethylformamide (1 ml), Tris pH 8.2 (49), IM Levamasole (50 μl) and Fast blue (50 mg).

The substrate mixture was added to each cytospin and allowed to develop for 15 mins followed by washing in distilled water and then mounted.

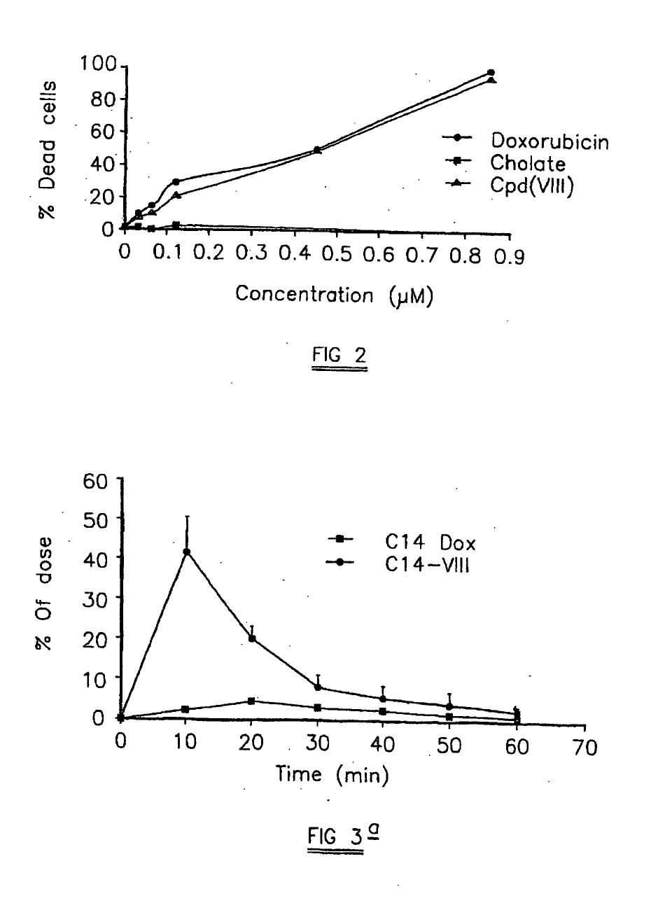

The results obtained are set out in Table 1 below and in accompanying FIG. 2.

TABLE 1
Toxicity of Compound (VIII) compared with that of doxorubicin and
cholate (% of dead cells)

| | | | | | Mean % |
|---|---|---|---|---|---|
| Conc'n. | Mean % | Conc'n. | Mean % | Conc'n. | Cpd |
| (μM) | Cholate | (μM) | Doxorubicin | (μM) | (VIII) |
| 0 | 1 | 0 | 1 | 0 | 0 |
| — | — | — | — | 0.00 | 3.3 |
| 0.03 | 1 | 0.03 | 9.4 | 0.03 | 7.6 |
| 0.06 | 0 | 0.06 | 14.9 | 0.06 | 10.8 |
| 0.12 | 1.8 | 0.12 | 28.9 | 0.12 | 20.5 |
| — | — | 0.45 | 50 | 0.45 | 49 |
| 0.86 | 1 | 0.86 | 100 | 0.86 | 96.6 |

It will be seen from the above Table that compound (VIII) has a similar toxicity to that of free doxorubicin at concentrations in the range of 0.06 to 0.86 μM.

In Vivo Biliary Excretion Tests

Cholyl-Lysyl-doxorubicin (VIII), free doxorubicin and free cholate were also subjected to biliary excretion tests as described below.

All animal studies were conducted in accordance with local institutional guidelines for the care and use of laboratory animals. Biliary excretion of ¹⁴C-Doxorubicin (¹⁴C-Dox) and ¹⁴C-Cholyllysyldoxorubicin (¹⁴C-CpdVIII) was determined in Wista rats. Rats weighing 250-300 g were anaesthetised with pentobarbital adminstered intraperitoneally (i.p. 50 mg/kg body wt). Following Iaparotomy, the common bile duct was cannulated in the upper half wit Portex polythene tubing (20 cm long, i.d.=0.29 mm, o.d.=61 mm, A. R. Horwell, London UK). The body temperature of the animals was monitored by rectal probe and maintained at 37.5±0.5° C. by constant temperature regulator. The animals (n=6) were then injected via the right jugular vein with either a bolus ¹⁴C-Dox (1 mg/300 μl) or ¹⁴C-Cpd VIII (2 mg/300 μl) in physiological saline. Every 10 min, aliquots of bile were collected for a total of 60 min. After 60 min, heart, liver, blood and urine samples were collected for radioactive analysis for their control of either ¹⁴C-Dox or ¹⁴C-Cpd VIII. All bile aliquots were also analysed for radioactivity in a Scintillation Count against a background of basal bile.

The results obtained are expressed as a percentage of the radioactivity in the dose administered and illustrated in Tables 2 and 3a below:

TABLE 2
Biliary excretion

| | Mean % | SD | Mean % | SD |
|---|---|---|---|---|
| Time | C14- | C14- | C14- | C14- |
| (min) | Doxorubicin | Doxorubicin | Cpd(VIII) | Cpd(VIII) |
| 0 | 0 | 0 | 0 | 0 |
| 10 | 2.3 | 0.3 | 41.7 | 9.7 |
| 20 | 4.7 | 0.7 | 20 | 3.1 |
| 30 | 2.6 | 0.4 | 8 | 3.5 |
| 40 | 2.4 | 0.4 | 5.6 | 2.4 |
| 50 | 1 | 0.1 | 4 | 2.1 |
| 60 | 0.8 | 0.1 | 2.5 | 0.9 |

TABLE 3a
Cumulative biliary excretion in Wistar rats

| Time | Mean % Dose | SD | Mean % Dose | SD |
|---|---|---|---|---|
| (min) | doxorubicin | Doxorubicin | Cpd (VIII) | Cpd (VIII) |
| 0 | 0 | 0 | 0 | 0 |
| 10 | 2.3 | 0.5 | 41.7 | 9.7 |
| 20 | 7 | 1.2 | 61.7 | 6.4 |
| 30 | 9.6 | 0.8 | 69.7 | 5.3 |
| 40 | 11.7 | 1 | 75.3 | 4.07 |
| 50 | 12.7 | 1.1 | 79.3 | 4.2 |
| 60 | 13.5 | 0.6 | 81.8 | 3.9 |

The results are also illustrated in accompanying FIGS. 3a and 3b.

It will be seen from the above that Compound (VIII) is rapidly directed to the hepatic/biliary system as compared with free doxorubicin

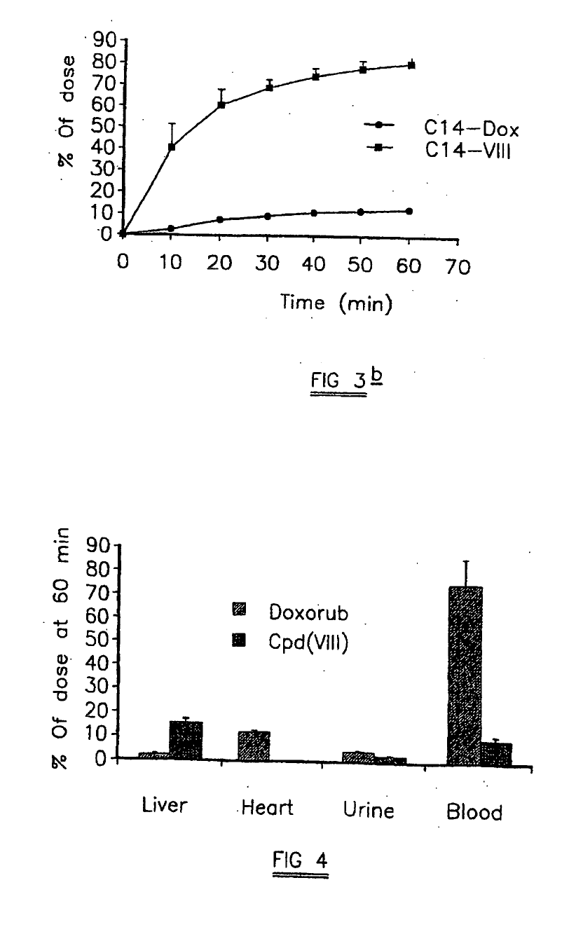

Further tests were performed to estimate the percentage of the dose of the compounds being tested in the liver, heart, urine and blood of Wistar rats. The results are illustrated in Table 3b and accompanying FIG. 4

TABLE 3b
Percentage of dose at 60 min

| | Mean % dose | SD | Mean % dose | SD |
|---|---|---|---|---|
| | Doxorubicin | Doxorubicin | Cpd (VIII) | Cpd (VIII) |
| Liver | 1.9 | 0.4 | 14.7 | 2 |
| Heart | 10.8 | 1 | 0 | 0 |
| Urine | 4 | 0.8 | 2.2 | 0.4 |
| Blood | 74.7 | 9.6 | 8.9 | 1.6 |
| Total | 91.4 | — | 25.8 | — |

It will be seen from Tables 3a and 3b that, after 60 minutes following administration, Compound (VIII) is effectively targeted to the liver, whilst free doxorubicin is to be found mainly in the blood and the heart and scarcely at all in the liver.

### EXAMPLE 2

Example 1 is repeated but using 3.568 mmol (0.879 g) of N-ε-tBOC-L-lysine instead of N-ε-F_{MOC}-L-lysine in Step 1 to produce cholyl-lysine-N-ε-tBOC which is then used (26 mg, 40 μM) in Step 2 to produce cholyl-lysyl(tBOC)doxorubicin which is then cleaved using 3M HCl in ethyl acetate instead of 5% piperidine as described in Step 2 of Example 1 to produce Compound (VIII).

### EXAMPLE 3

Example 1 or 2 is repeated using an equivalent amount of deoxycholic acid in place of the cholic acid to produce a compound of the formula (X):

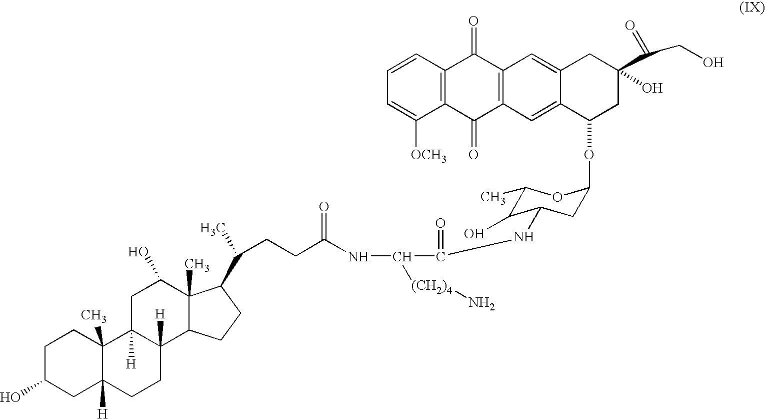

### EXAMPLE 4

Example 1 or 2 is repeated using an equivalent amount of lithocholic acid in place of the cholic acid to produce a compound of the formula (X):

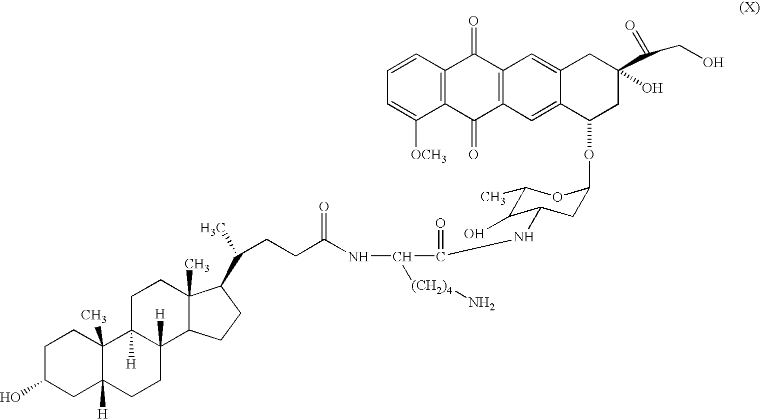

### EXAMPLE 5

Doxorubicin hydrochloride (24 mg, 40 μmol) and triethylamine (6 μl, 40 μmol) were suspended in 300 μl of N,N-dimethylformamide (DMF). Ursodeoxycholyl-lysyl-F_{MOC} (34 mg, 40 μl) was added. After cooling to 0-2° C,, 1-hydroxybenzotriazole (6 mg, 40 μmol) was added followed by dicyclohexylcarbodiimide (8 mg, 40 μmol) in 100 μl DMF. The mixture was stirred for 30 minutes at 0° C. and for 18 hours at room temperature (21° C.) in the dark. At the end of the reaction, the mixture was filtered and methanol (400 μl) was added to the filtrate followed by addition of diisopropylether to precipitate ursodeoxycholyl-lysyl--doxorubicin-F_{MOC} (UCLDFmoc). The precipitate was washed 3 times with diisopropyl ether and then dissolved in chloroform (1 ml) followed by precipitation from diethylether to give UCLDFmoc in a yield of 90-96% and a purity range of 91-94%.

Mild cleavage of 6 mg of UCLDFmoc was performed using piperidine (5 μl) in 200 μl DMP (i.e. 2.5% v/v piperidine) with stirring for 5.5 minutes at room temperature to produce ursodeoxycholyl-lysyl-doxorubicin (yield 89-92% at a purity of 90-92%).

### EXAMPLE 6

Example 5 was repeated using deoxycholyl-lysyl-F_{MOC} (30.6 mg, 40 μl) instead of ursodeoxycholyl-lysyl-F_{MOC} (30.6 mg, 40 μl) to give deoxycholyl-lysyl-doxorubicin yield 89-92% at a purity of 90-92%).

### EXAMPLE 7

Example 5 was repeated using hyocholyl-lysyl-F_{MOC} (34 mg, 40 μl) of ursodeoxycholyl-lysyl-F_{MOC} (30.6 mg, 40 μl) to give hyocholyl-lysyl-doxorubicin (yield 89-92% at a purity of 9-92%).

### EXAMPLE 8

Example 5 was repeated using lithocholyl-lysyl-F_{MOC} (30 mg, 40 μl) instead of ursodeoxycholyl-lysyl-F_{MOC} (30.6 mg, 40 μl) to give lithocholyl-lysyl-doxorubicin (yield 89-92% at a purity of 90-92%).

### EXAMPLE 9

10 mmol (3.72 g) Tamoxifen (1-(p-β-dimethylaminoethoxyphenyl)-trans-1,2-diphenylbut-1-ene) in N,N-dimethylformamide (DMF) or tetramethylene sulfone (8 ml) under anhydrous conditions is added to a solution of 0.4 g nitronium tetrafluoroborate in 6 ml of DMF or tetramethylene sulfone with stirring at such a rate that the temperature is maintained between 15° and 25° C. After the addition has been completed, stirring is continued between 20° and 35° C. for 30 min. The reaction mixture is then cautiously poured into 5 ml of cold water. The organic layer is separated, washed twice with water, and dried over calcium chloride. The excess aromatic substrate is distilled off under reduced pressure to yield Tamoxifen nitrate.

The Tamoxifen nitrate is then reacted with stannous chloride (Sncl₂) in acid (H₃O⁺) followed by addition of sodium hydroxide solution to give Tamoxifenamine, yield 92%. The resultant Tamoxifenamine is then reacted with cholyl-lysine-N-ε-Fmoc or cholyl-lysin-N-ε-tBOC in an analogous manner to that described in Example 1 or 2 above for the linking of doxorubicin with cholyl-lysineF_{MOC} or cholyl-lysine-tBOC. Mild cleavage using 5% piperidine yields cholyllysltamoxifen which is subjected to sulphonation to yield the cholyllysltamoxifen sulphate which is soluble in water.

### COMPARATIVE EXAMPLE

### Synthesis of CholyllysylCBZDoxorubicin (CL(CBZ)-Dox)

Doxorubicin hydrochloride (48 mg. 80 μmol) and triethylamine (12 μl, 80 μmol) were suspended in 600 μl N,N-dimethylformamide (DMF). Cholyllsine-N-ε-CBZ (64 mg 80 μmol) dissolved in 0.4 ml DMF was added. After cooling to 0-2° C., 1-hydroxybenzotriazol (12 mg. 80 μmol) was added, followed by dicyclohexylcarbodiimide (16 mg. 80 μmol) in 0.2 ml DMF. The mixture was stirred for 30 min at 0° C. and for 18 h at room temperature (21° C.) in the dark. The DMF solution formed was acidified with 200 μl dilute ethanoic acid solution. After centrifugation, the supernatant was collected and CL(CBZ)-Dox was precipitated by addition of ethyl acetate. The precipitate was dried to obtain CL(CBZ)-Dox in 92% yield.

Cytotoxicity Assay

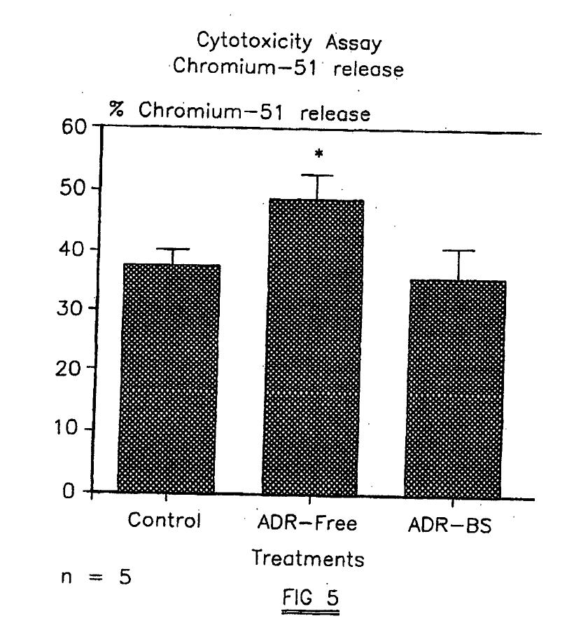

Referring now to FIG. 5, there are shown the results obtained in cyotoxicity tests conducted by incubating monolayer cultures of rat hepatocytes with chromium-51 (4 uCi) for 18 hours, followed by incubation with 0.012M CL(CBZ) and CL(CBZ)Dox. Activities of supernatants and cells were measured. Chromium-51 leakage is expressed as a percentage of the total activity (medium+cells). Values are mean±SD [n=5]. *=P<0.001 CL(CBZ)/CL(CBZ)-Dox and a control (no material added after incubation with chromium-51).

No significant differences (P>0.1) were observed between the control (37.61±1.56%), CL(CBZ)-Dox (36.45±3.77%) and CL(CBZ) (49.01±2.57%). This shows that it is important to provide a free amino group to replace the amino group of doxorubicin which has been used to form the amide group in the linkage with the cholyl-lysyl moiety.

Further Cytotoxicity Tests

Cholyl-lysyl-doxorubicin (VIII) was investigated for antiproliferative activity in vitro in 12 human tumour cell lines (including the large cell lung carcinoma H460, the mammary carcinoma cell line MCF7, the uterus carcinoma cell line UXF 1138L and the large cell lung carcinoma LXFL 529L), and was compared to free doxorubicin and cholic acid as controls. 5-20.000 cells/well were plated into 96 wells. After 24 hours, the compounds were added in do levels from 3 nM to 30 μM (cholyl-lysyl-doxorubicin and cholic acid) and from 0.3 nM to 3 μM (doxorubicin), respectively. After 4 days of continuous exposure to the test compound, cellular DNA content was determined using propidium iodide, which results in fluorescence signals that correlate with cell number.

Cholyl-lysyl-doxorubicin was found to have a potential activity similar to doxorubicin. The mean IC₇₀ of cholyl-lysyl-doxorubicin was 1.3 μM compared to 0.7 μM for free doxorubicin. The cholyl-lysyl-doxorubicin and the free doxorubicin showed differential patterns of cytotoxicity, but the tumour selectivities of these compounds were nearly identical. The most sensitive cell lines to cholyl-lysyl-doxorubicin were the large cell lung carcinoma H460, the mammary carcinoma cell line MCF7, the uterus carcinoma cell line UXF 1138L and the large cell lung carcinoma LXFL 529L. The free cholic acid showed no antitumour activity in vitro.

1. A liver-targeting active compound having the general formula (I): 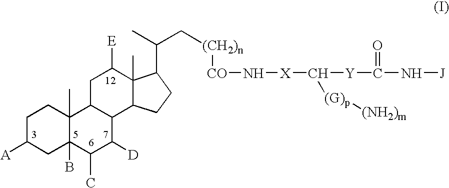
wherein A is α-OH or β-OH, B is α-H or β-H, C is —H, α-OH or , β-OH, or B and C together form a double bond, D is —H, α-OH or β-OH, E is —H, α-OH or β-OH, -G- is a side chain moiety, —NH-J is selected from (i) a residue of an amino group-containing active compound wherein said —NH— group is provided by said amino group of the active compound, and (ii) a residue of an active compound to which an amino group has been added wherein said —NH— group is provided by said added amino group; each of X and Y independently represents a single bond, —(CH₂)_{z}— (where z is 1 to 8), —O— or —S—; n is 0 or 1; m is 0 or 1; and p is 0 or 1; provided that, when —NH-J is (i), m is 1.

2. A compound as claimed in claim 1, wherein —NH-J is said residue of an amino group-containing active compound wherein said —NH— group is provided by said amino group of the active compound, and wherein m is 1.

3. A compound as claimed in claim 1, wherein —NH-J is a residue of an active compound to which an amino group has been added and wherein said —NH— group is provided by said added amino.

4. A compound as claimed in claim 1, wherein -G- is —(CH₂)_{q}—, —O— or —S—, wherein q is between 1 to 8.

5. A compound as claimed in claim 4, wherein -G- is —(CH₂)_{q}— where q is 1 to 5.

6. A compound as claimed in claim 5, wherein q is 3 to 5.

7. A compound as claimed in claim 5, wherein q is 4.

8. A compound as claimed in claim 1, wherein each of X and Y independently represents a single bond or —(CH₂)_{z}.

9. A compound as claimed in claim 9, wherein each of X and Y independently represents a single bond or —(CH₂)_{z}, where z is I to 5.

10. A compound as claimed in claim 1, wherein p is 1, -G- is —O—, —S— or —(CH₂)_{q}— (-where q is 3 to 5-), and Y represents a single bond.

11. A compound as claimed in claim 1, wherein —NH-J is based on a pharmaceutically active compound selected from the group consisting of doxorubicin; epirubicin; mitoxantrone; methotrexate; tamoxifen; mitomycin C; fluorouracil; cytarabine; thioguanine; acyclovir; ganciclovir; amphotericin; primaquine; ursodeoxycholyllysylcysteine; ursodeoxycholyllysylcysteic acid; ursodeoxycholyllysylmethionine; ursodeoxycholyllysyl-glutathione-(reduced); ursodeoxycholyllysyll-methionine sulfone; amethopterin; arabinosyl-cytosine; L-cysteic acid; cysteine; L-cysteine sulphinic acid; N-acetylcysteine; methionine; methionine sulphone; methionine sulphoxide; L-glutathione; S-adenosyl-homocysteine; S-adenosyl-methionine; 8-aminoquinoline; tilorone(2,7-bis[2-(diethylamino)ethoxy]-9H-fluoren-9-one); tilorone analogs such as tilorone dihydrochloride, 3,6-bis[2-(dimethylamino)ethoxy]-9H-xanthen-9-one dihydrochloride, 2,7-bis[dimethylaminoacetyl]-9H-xanthene dihydrochloride hydrate, 2,8-bis[dimethylaminoacetyl]-dibenzothiophene dihydrochloride hydrate and 2,8-bis[dimethylaminoacetyl]-dibenzofuran dihydrochloride hydrate; melphalan(4-(bis[2-chloroethyl]amino)-L-phenylalanine, peptides, proteins and nucleotides.

12. A compound as claimed in claim 1, wherein the pharmaceutically active compound is selected from doxorubicin and tamoxifen.

13. A compound as claimed in claim 1, further comprising a steroid moiety selected from the group consisting of cholic acid, chenodeoxycholic acid, deoxycholic acid, hyodeoxycholic acid, hyocholic acid, α-, β-, or ω-muricholic, acid, a nor-bile acid, lithocholic acid, 3β-hydroxycholenoic acid, ursodeoxycholic acid, and allocholic acid (5α-cholan-24-oic acid).

14. A compound as claimed in claim 13, wherein the steroid moiety is selected from cholic acid, deoxycholic, acid, lithocholic acid, ursodeoxycholic and hyocholic acid.

15. A compound of the general formula (III): 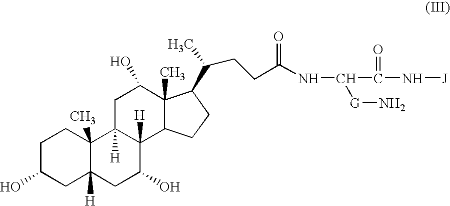
where G and J are as defined in claim 1.

16. A compound as claimed in claim 15, wherein the residue —NH-J is based on doxorubicin.

17. A compound as claimed in claim 15, wherein G is —O—, —S— or —(CH₂)_{q}—, wherein q is between 1 to 8.

18. A method of preparing a compound of the general formula (1) as defined in claim 1, comprising the step of reacting a compound of the general formula (II): 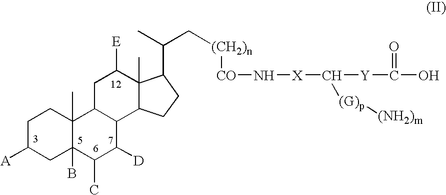
(wherein A, B, C, D, E, G, n, m and p are as defined in claim 1) with an active compound having an amino group, so as to form an amide linkage between the carboxyl group in the compound of the general formula (II) and the amino group of the active compound.

19. A compound as claimed in claim 15, wherein G is —(CH₂)_{q}— and q is 1 to 5.

20. A compound as claimed in claim 15, wherein G is —(CH₂)_{q}— and q is 3 to 5.

21. A compound as claimed in claim 15, wherein G is —(CH₂)_{q}— and q is 4.
